(19) United States
(12) Patent Application Publication
(10) Pub. No.: US 2024/0324890 A1
(43) Pub. Date: Oct. 3, 2024

(54) INFORMATION PROCESSING APPARATUS, INFORMATION PROCESSING METHOD, AND PROGRAM

(71) Applicant: SONY GROUP CORPORATION, Tokyo (JP)

(72) Inventors: Koki KATSUMATA, Tokyo (JP); Naoya SAZUKA, Tokyo (JP)

(73) Assignee: SONY GROUP CORPORATION, Tokyo (JP)

(21) Appl. No.: 18/576,501

(22) PCT Filed: Feb. 25, 2022
(86) PCT No.: PCT/JP2022/007808

§ 371 Date: Jan. 4, 2024

(30) Foreign Application Priority Data
Jul. 19, 2021 (JP) 2021-118408

(51) Int. Cl. A61B 5/0531; A61B 5/352

(52) U.S. Cl. CPC A61B 5/0531; A61B 5/352

(57) ABSTRACT

The present technology relates to an information processing apparatus, an information processing method, and a program that enable of improvement in detection accuracy of human physiological indicators. The information processing apparatus includes: a detection parameter setting unit that sets a detection parameter on the basis of at least one of a feature amount of an electrodermal activity signal indicating an electrodermal activity of a predetermined site of a user, biological information of the user, or environment information of the user, the detection parameter being used in a case of detecting a physiological indicator on the basis of the electrodermal activity signal; and a physiological indicator detection unit that detects the physiological indicator on the basis of the electrodermal activity signal and the detection parameter.

### TECHNICAL FIELD

The present technology relates to an information processing apparatus, an information processing method, and a program and more particularly relates to an information processing apparatus, an information processing method, and a program that are favorable to be used in a case of detecting physiological indicators of a user.

### BACKGROUND ART

Conventionally, an electrodermal activity (EDA) is used as a physiological indicator for estimating an internal status (e.g., emotion) of a human. The EDA is a signal electrically measured on the basis of skin conductance indicating the conductivity of current through the skin due to emotional sweating reflecting sympathetic nervous activity.

The EDA measured based on the skin conductance exhibits mainly two physiological indicators, a skin conductance level (SCL) and a skin conductance response (SCR). The SCL indicates a gentle change in sweating on the skin surface. The SCR indicates an instant change in sweating. More SCRs are generated when the human is conscious. SCRs are hardly generated when the human is relaxed. That is, the EDA is separated into the SCL with a slow movement on the baseline and the SCR with a quick movement on the baseline.

Although the EDA is typically measured at fingers and palm, it is desirable to measure it at a wrist to which electrodes can be attached easier than the fingers and palm. However, the wrist is unlikely to exhibit the EDA because the wrist has density of sweat glands lower than the finger tip and palm. Therefore, a small change in internal status may not be detected or a large change in internal status may be detected with a delay in a case of estimating the human internal status on the basis of the EDA measured at the wrist.

In this regard, improving SCR detection sensitivity by amplifying the SCR on the basis of the SCL by the use of the SCR dependency on the SCL has been conventionally proposed (e.g., see Patent Literature 1).

### CITATION LIST

### Patent Literature

Patent Literature 1: WO 2021/106551

### DISCLOSURE OF INVENTION

### Technical Problem

However, the invention according to Patent Literature 1 may amplify not only the SCR but also noise.

The present technology has been made in view of such circumstances and enables improvement in detection accuracy of human physiological indicators.

### Solution to Problem

An information processing apparatus according to an aspect of the present technology includes: a detection parameter setting unit that sets a detection parameter on the basis of at least one of a feature amount of an electrodermal activity signal indicating an electrodermal activity of a predetermined site of a user, biological information of the user, or environment information of the user, the detection parameter being used in a case of detecting a physiological indicator on the basis of the electrodermal activity signal; and a physiological indicator detection unit that detects the physiological indicator on the basis of the electrodermal activity signal and the detection parameter.

An information processing method according to an aspect of the present technology includes: setting a detection parameter on the basis of at least one of a feature amount of an electrodermal activity signal indicating an electrodermal activity of a predetermined site of a user, biological information of the user, or environment information of the user, the detection parameter being used in a case of detecting a physiological indicator on the basis of the electrodermal activity signal; and detecting the physiological indicator on the basis of the electrodermal activity signal and the detection parameter.

A program according to an aspect of the present technology includes: setting a detection parameter on the basis of at least one of a feature amount of an electrodermal activity signal indicating an electrodermal activity of a predetermined site of a user, biological information of the user, or environment information of the user, the detection parameter being used in a case of detecting a physiological indicator on the basis of the electrodermal activity signal; and detecting the physiological indicator on the basis of the electrodermal activity signal and the detection parameter.

In an aspect of in the present technology, a detection parameter is set on the basis of at least one of a feature amount of an electrodermal activity signal indicating an electrodermal activity of a predetermined site of a user, biological information of the user, or environment information of the user, the detection parameter being used in a case of detecting a physiological indicator on the basis of the electrodermal activity signal, and the physiological indicator is detected on the basis of the electrodermal activity signal and the detection parameter.

### BRIEF DESCRIPTION OF DRAWINGS

FIG. 1 A block diagram showing an embodiment of a wearable device to which the present technology is applied.

FIG. 2 A block diagram showing configuration examples of a control unit of the wearable device.

FIG. 3 A flowchart for describing physiological indicator detection processing.

FIG. 4 A graph that compares EDA signals measured at a wrist and a finger tip.

FIG. 5 A graph showing a relationship between a curvature of an SCL signal and a detection delay of a conscious reaction.

FIG. 6 A graph that compares SCR signals in a case where an SCR discriminative threshold is fixed and in a case where the SCR discriminative threshold is adjusted.

FIG. 7 A diagram for describing a method of setting the SCR discriminative threshold on the basis of a distribution of impulse amplitudes of impulse responses.

FIG. 8 A diagram for describing the method of setting the SCR discriminative threshold on the basis of a distribution of impulse amplitudes of impulse responses.

### MODE(S) FOR CARRYING OUT THE INVENTION

Hereinafter, modes for carrying out the present technology will be described. The descriptions will be made in the following order.
1. Embodiment
2. Modified Examples
3. Effects of Present Technology
4. Others

### 1. EMBODIMENT

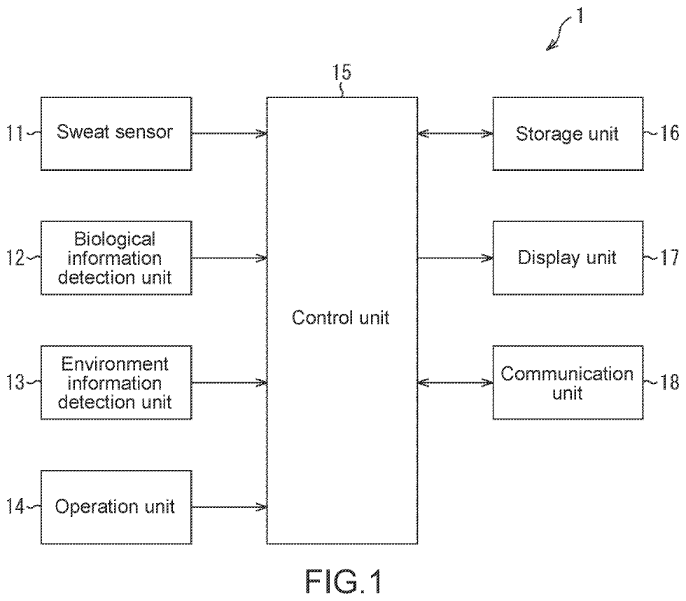

First of all, an embodiment of the present technology will be described with reference to FIGS. 1 to 6.

### Configuration Example of Wearable Device 1

FIG. 1 shows an embodiment of a wearable device 1 to which the present technology is applied.

The wearable device 1 is, for example, a watch-type (wristband-type) and is wrapped around the wrist of a user for use. The wearable device 1 is a device that detects skin conductance of the user's wrist as an electrodermal activity (EDA) and detects a skin conductance response (SCR) and a skin conductance level (SCL) for example on the basis of a measurement result of the EDA.

The wearable device 1 includes a sweat sensor 11, a biological information detection unit 12, an environment information detection unit 13, an operation unit 14, a control unit 15, a storage unit 16, a display unit 17, and a communication unit 18.

The sweat sensor 11 detects skin conductance of a skin surface of the user's wrist as the EDA and supplies an EDA signal indicating a detection result (skin conductance) of the EDA to the control unit 15.

The biological information detection unit 12 includes various biological sensors. The biological information detection unit 12 detects biological information of the user on the basis of a detection result of each biological sensor. For example, the biological information detection unit 12 detects pulse waves, a blood flow, an electrocardiogram, a ballistocardiogram, and the like of the user through the biological sensors and detects, on the basis of the detection results, a heart rate (HR), heart rate variability (HRV), and the like as the biological information. The biological information detection unit 12 supplies the detected biological information to the control unit 15.

The environment information detection unit 13 includes various sensors and detects environment information indicating an environment of the user on the basis of the detection results of the respective sensors. The environment information includes information indicating status and action (context) of the user, an environment around the user, and the like. Specifically, the environment information includes, for example, acceleration and angular velocity of the wearable device 1 (e.g., in vicinity of the user's wrist), audio data indicating the user's voice or sounds around the user, and image data obtained by imaging the user or the periphery of the user. Moreover, for example, the environment information includes information regarding the user's action (e.g., sleeping, meeting, walking, etc.) estimated on the basis of such information. The biological information detection unit 12 supplies the detected biological information to the control unit 15.

The operation unit 14 is various operation units, e.g., press-type and proximity-type operation units. The operation unit 14 detects an operation made by the user and supplies operation information indicating a detection result to the control unit 15. It should be noted that the operation unit 14 may include a proximity sensor provided on the display unit 17.

The control unit 15 executes various arithmetic operations on the basis of various programs stored in the storage unit 16 and comprehensively controls the respective portions of the wearable device 1. Moreover, the control unit 15 detects physiological indicators of the user on the basis of at least one of the EDA signal, the biological information, or the environment information.

The control unit 15 is realized by hardware or a combination of hardware and software. The hardware is configured as a part or whole of the control unit 15. The hardware can be, for example, a central processing unit (CPU), a digital signal processor (DSP), a field programmable gate array (FPGA), an application specific integrated circuit (ASIC), or a combination of two or more of them.

The storage unit 16 includes various programs necessary for the processing of the control unit 15, a nonvolatile memory for storing various data, and a volatile memory used for a working area for the control unit 15.

The display unit 17 is constituted by, for example, a liquid-crystal display or an electro luminescence (EL) display. The display unit 17 is controlled by the control unit 15 to display various images and the like, for example, the current time, icons indicating various applications such as music, game, mail, and browser, and videos, still images, and the like associated with the executed applications.

The communication unit 18 is configured to be capable of communicating with another apparatus. Examples of the other apparatus capable of communicating with the wearable device 1 can include various personal computers such as a desktop personal computer, a mobile phone (including a smartphone), and a server apparatus on a network.

### Configuration Example of Control Unit 15

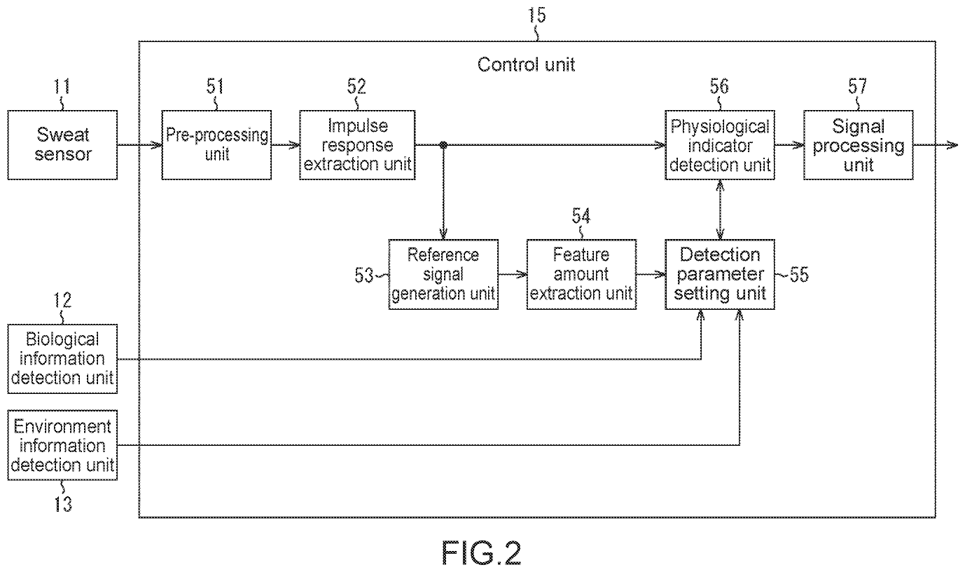

FIG. 2 shows some of configuration examples of the control unit 15 of the wearable device 1. FIG. 2 mainly shows configuration examples of a portion associated with detection processing of physiological indicators of the control unit 15.

The control unit 15 includes a pre-processing unit 51, an impulse response extraction unit 52, a reference signal generation unit 53, a feature amount extraction unit 54, a detection parameter setting unit 55, a physiological indicator detection unit 56, and a signal processing unit 57.

The pre-processing unit 51 performs predetermined pre-processing (e.g., a band-pass filter) on the EDA signal supplied from the sweat sensor 11 and performs preparation at a pre-stage to extract an impulse response. The pre-processing unit 51 supplies the pre-processed EDA signal to the impulse response extraction unit 52.

The impulse response extraction unit 52 performs impulse response extraction processing on the pre-processed EDA signal. The impulse response extraction unit 52 supplies an impulse response row in which extracted impulse responses are arranged in a time series and the EDA signal to the reference signal generation unit 53 and the physiological indicator detection unit 56.

The reference signal generation unit 53 generates an SCR signal for reference (hereinafter, referred to as a reference SCR signal) and an SCL signal for reference (hereinafter, referred to as a reference SCL signal), for example, on the basis of the impulse response row. The reference signal generation unit 53 supplies the reference SCR signal, the reference SCL signal, and the impulse response row to the feature amount extraction unit 54.

The feature amount extraction unit 54 extracts a feature amount of the EDA signal on the basis of the reference SCR signal, the reference SCL, and the impulse response row. The feature amount extraction unit 54 supplies the detection parameter setting unit 55 with information indicating an extraction result of the feature amount of the EDA signal.

The detection parameter setting unit 55 sets a detection parameter to be used for detecting physiological indicators of the user on the basis of at least one of the feature amount of the EDA signal, the biological information of the user, or the environment information of the user. The detection parameter setting unit 55 supplies information indicating the set detection parameter to the physiological indicator detection unit 56.

The physiological indicator detection unit 56 detects physiological indicators of the user on the basis of the impulse response row and the detection parameter. The physiological indicator detection unit 56 supplies information indicating a detection result of the physiological indicators to the detection parameter setting unit 55 and the signal processing unit 57. Moreover, the physiological indicator detection unit 56 supplies the EDA signal to the signal processing unit 57.

The signal processing unit 57 performs predetermined signal processing on the basis of the detection result of the physiological indicators and the EDA signal and generates an output signal indicating the detection result of the physiological indicators. The signal processing unit 57, for example, stores the output signal in the storage unit 16, supplies it to the display unit 17, or sends it to the other apparatus via the communication unit 18.

### <Physiological Indicator Detection Processing>

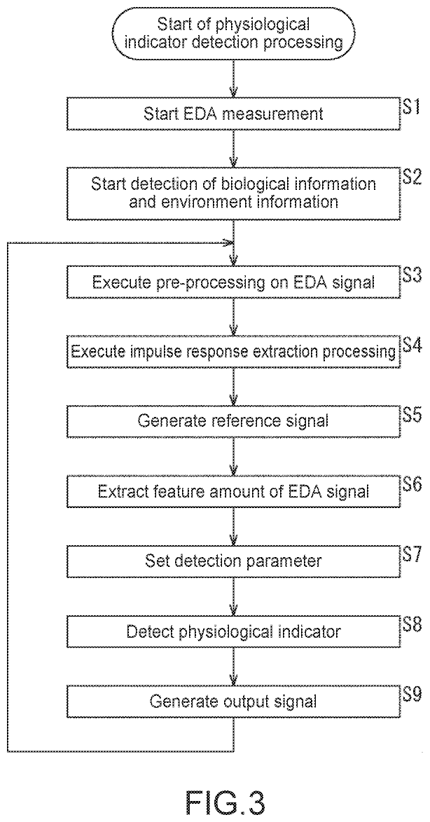

Next, physiological indicator detection processing executed by the wearable device 1 will be described with reference to the flowchart in FIG. 3.

This processing is started when the wearable device 1 is powered on and ends when the wearable device 1 is powered off, for example.

In Step S1, the sweat sensor 11 starts measurement of the EDA. Specifically, the sweat sensor 11 starts processing of detecting skin conductance of the skin surface of the user's wrist as the EDA and supplying an EDA signal indicating a detection result of the EDA to the pre-processing unit 51.

In Step S2, the biological information detection unit 12 and the environment information detection unit 13 start detection of biological information and environment information.

Specifically, the biological information detection unit 12 starts processing of detecting biological information of the user on the basis of detection results of the respective biological sensors and supplying the detected biological information to the control unit 15. Examples of the biological information includes HR and HRV of the user.

The environment information detection unit 13 starts processing of detecting environment information of the user on the basis of the detection results of the respective sensors and supplying the detected environment information to the control unit 15. The environment information includes, for example, acceleration and angular velocity of the wearable device 1 (e.g., in vicinity of the user's wrist), audio data indicating the user's voice or sounds around the user, and image data obtained by imaging the user or the periphery of the user, and information regarding the user's action.

In Step S3, the pre-processing unit 51 executes pre-processing on the EDA signal. For example, the pre-processing unit 51 executes pre-processing such as a band-pass filter on the EDA signal supplied from the sweat sensor 11. The pre-processing unit 51 supplies the pre-processed EDA signal to the impulse response extraction unit 52.

In Step S4, the impulse response extraction unit 52 executes impulse response extraction processing. For example, the impulse response extraction unit 52 executes the impulse response extraction processing in accordance with an approach according to “Benedek, M. & Kaernbach, C. (2010). Decomposition of skin conductance data by means of nonnegative deconvolution. Psychophysiology, 47, 647-658” (hereinafter, referred to as Cited Document 1). Specifically, for example, the impulse response extraction unit 52 subjects the pre-processed EDA signal to a deconvolution arithmetic operation using the Bateman function as a transfer function, thereby extracting an impulse response. The impulse response extraction unit 52 supplies an impulse response row in which the extracted impulse responses are arranged in a time series and the EDA signal to the reference signal generation unit 53 and the physiological indicator detection unit 56.

In Step S5, the reference signal generation unit 53 generates a reference signal. For example, the reference signal generation unit 53 separates the respective impulse responses included in the impulse response row by using a predetermined SCR discriminative threshold into an impulse response associated with the SCR (hereinafter, referred to as an SCR impulse response) and an impulse response associated with noise and the like other than the SCR (hereinafter, referred to as a noise impulse response). The reference signal generation unit 53 generates a reference SCR signal by executing a convolution arithmetic operation corresponding to the deconvolution arithmetic operation performed in the processing of Step S4 on the extracted SCR impulse response group. The reference signal generation unit 53 generates a reference SCL signal by taking a difference between the EDA signal and the SCR signal. Accordingly, the EDA signal is separated into the reference SCR signal and the reference SCL signal. The reference signal generation unit 53 supplies the reference SCR signal, the reference SCL signal, and the impulse response row to the feature amount extraction unit 54.

In Step S6, the feature amount extraction unit 54 detects a feature amount of the EDA signal.

For example, the feature amount extraction unit 54 calculates a curvature of the reference SCL signal in a previous predetermined period of time (e.g., 60 seconds) (hereinafter, referred to as a curvature determination period) as the feature amount of the EDA signal. For example, a tilt of the curve of the reference SCL signal calculated with the first derivative of the curve of the reference SCL signal is calculated as the curvature of the reference SCL signal.

For example, the feature amount extraction unit 54 calculates a level of the reference SCL signal as the feature amount of the EDA signal. For example, an average value of values of the reference SCR signal in the curvature determination period is calculated as the level of the reference SCL signal.

For example, the feature amount extraction unit 54 calculates a rise time constant of each of the impulse responses included in the impulse response row as the feature amount of the EDA signal.

The feature amount extraction unit 54 supplies information indicating an extraction result of the feature amount of the EDA signal to the detection parameter setting unit 55.

In Step S7, the detection parameter setting unit 55 sets a detection parameter. For example, the detection parameter setting unit 55 sets a SCR discriminative threshold for discriminating the SCR impulse response from the impulse responses included in the impulse response row, as the detection parameter.

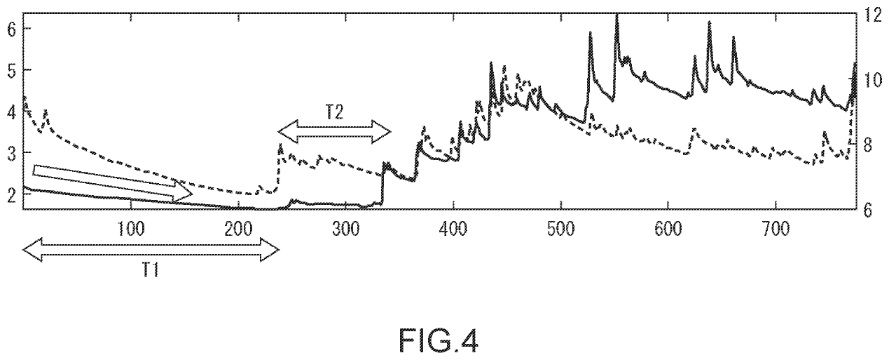

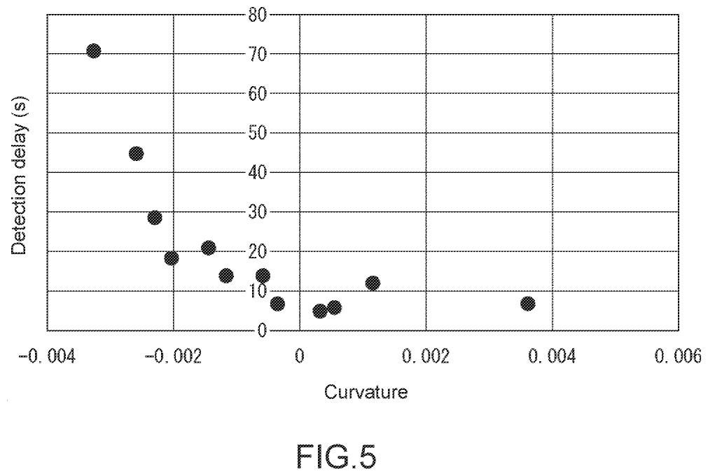

Here, an example of a method of setting the SCR discriminative threshold will be described with reference to FIGS. 4 and 5.

FIG. 4 is a graph showing an example that compares an EDA signal measured at the user's wrist (hereinafter, referred to as a wrist EDA signal) with an EDA signal measured at the finger tip of the user (hereinafter, referred to as a finger tip EDA signal). The graph of the wrist EDA signal is shown as the solid line and the graph of the finger tip EDA signal is shown as the dotted line. The horizontal axis indicates time (seconds), the left vertical axis indicates skin conductance (μS) of the wrist EDA signal, and the right vertical axis indicates skin conductance (μS) of the finger tip EDA signal.

Here, effects of the sympathetic nervous system are dominant in the EDA. Thus, the reaction speed of the EDA increases when the user is conscious and the reaction speed of the EDA lowers when the user is relaxed. Thus, in a recovery period in which recovery from the conscious reaction is done, the SCL signal included in the EDA signal gently decreases and the curvature of the SCL signal is negative.

Moreover, in a case where the conscious reaction has occurred during the recovery from the conscious reaction, the reaction strength of the EDA with respect to the conscious reaction is smaller than in a case where the conscious reaction has occurred at the relax time. In addition, the longer the recovery period, the weaker the reaction strength of the EDA with respect to the conscious reaction. Thus, the amplitude of the SCR signal included in the EDA signal becomes low just after the conscious reaction occurs during the recovery period.

For example, as shown in FIG. 4, in a recovery period T1, (the SCL signal included in) the wrist EDA signal and (the SCL signal included in) the finger tip EDA signal gently decrease and the curvature is negative.

Moreover, in a case where the conscious reaction has occurred just after the recovery period T1, the change rate (amplitudes of the SCR signals) of the wrist EDA signal and the finger tip EDA signal become lower than that in the subsequent period. In particular, in a period T2 just after the recovery period T1, the change rate of the wrist EDA signal becomes significantly lower than the change rate of the finger tip EDA signal and the SCR can be hardly detected from the wrist EDA signal.

Accordingly, in a case of detecting a conscious reaction of the user on the basis of the SCR of the wrist EDA signal, a detection delay of the period T2 can be generated as compared to a case of detecting a conscious reaction of the user on the basis of the SCR of the finger tip EDA signal.

FIG. 5 is a graph showing an example of a relationship between the curvature of the SCL signal included in the wrist EDA signal just before the conscious reaction occurs and the detection delay of the conscious reaction. The horizontal axis in FIG. 5 indicates curvature of the SCL signal. The vertical axis in FIG. 5 indicates a detection delay (seconds) in a case of detecting the conscious reaction on the basis of the wrist EDA signal with respect to a case of detecting the conscious reaction on the basis of the finger tip EDA signal.

As it can be seen from the graph, the detection delay more easily occurs in a case where the curvature of the SCL signal just before the conscious reaction occurs is negative. As it can also be seen, in a case where the curvature of the SCL signal is negative, the detection delay time increases as the curvature of the SCL signal decreases (as the absolute value of the curvature of the SCL signal increases).

In this regard, for example, the detection parameter setting unit 55 sets the SCR discriminative threshold on the basis of the curvature of the reference SCL signal in the previous curvature determination period.

For example, the detection parameter setting unit 55 sets the SCR discriminative threshold as a default value in a case where the curvature of the reference SCL signal in the curvature determination period is positive or zero. For example, the SCR discriminative threshold is set to be 0.01 that is a default value according to Cited Document 1.

On the other hand, the detection parameter setting unit 55 sets the SCR discriminative threshold to be a value (e.g., 0.005) smaller than the default value in a case where the curvature of the reference SCL signal in the curvature determination period is negative. Accordingly, the detection sensitivity of the SCR increases.

The detection parameter setting unit 55 supplies information indicating the set SCR discriminative threshold to the physiological indicator detection unit 56.

In Step S8, the physiological indicator detection unit 56 detects physiological indicators. For example, the physiological indicator detection unit 56 separates the respective impulse responses into the SCR impulse response and the noise impulse response on the basis of amplitudes of the respective impulse responses (hereinafter, referred to as impulse amplitudes) included in the impulse response row supplied from the impulse response extraction unit 52 and the SCR discriminative threshold. For example, the impulse response whose impulse amplitude is equal to or higher than the SCR discriminative threshold is discriminated as the SCR impulse response and the impulse response whose impulse amplitude is lower than the SCR discriminative threshold is discriminated as the noise impulse response.

The physiological indicator detection unit 56 supplies an SCR impulse response row in which the extracted SCR impulse responses are arranged in a time series to the detection parameter setting unit 55 and the signal processing unit 57. Moreover, the physiological indicator detection unit 56 supplies the EDA signal to the signal processing unit 57.

In Step S9, the signal processing unit 57 generates an output signal.

For example, the signal processing unit 57 generates an output signal including the SCR impulse response row.

Alternatively, for example, the signal processing unit 57 generates an output signal indicating the number of SCR impulse responses in the previous predetermined period of time.

Alternatively, for example, the signal processing unit 57 generates an SCR signal by performing a convolution arithmetic operation corresponding to the deconvolution arithmetic operation performed in the processing of Step S4 on the SCR impulse response row. Moreover, the signal processing unit 57 generates the SCL signal by taking a difference between the EDA signal and the SCR signal. The signal processing unit 57 generates an output signal including the SCL signal and the SCR signal.

The signal processing unit 57, for example, stores the output signal in the storage unit 16, supplies it to the display unit 17, or sends it to the other apparatus via the communication unit 18.

Then, the processing returns to Step S3 and the processing of Steps S3 to S9 is repeatedly executed.

As described above, the SCR discriminative threshold is suitably adjusted and the detection sensitivity of the SCR is suitably adjusted on the basis of the curvature of the reference SCL signal. Accordingly, for example, in a case of using the wrist EDA signal for detecting a conscious reaction of the user, the occurrence of the detection delay of the conscious reaction is suppressed as compared to a case of using the finger tip EDA signal.

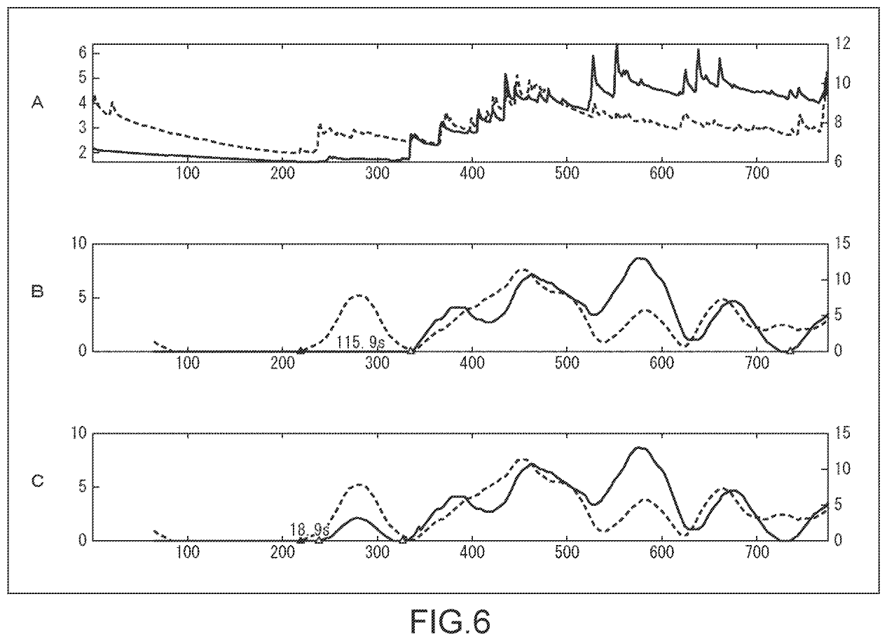

FIG. 6 shows an example that compares an SCR signal in a case where the SCR discriminative threshold is fixed with an SCR signal in a case where the SCR discriminative threshold is adjusted on the basis of the curvature of the reference SCL signal.

A of FIG. 6 shows a graph of the wrist EDA signal and the finger tip EDA signal similar to those of FIG. 4.

B of FIG. 6 shows a graph of the number of SCR impulse responses detected in a predetermined period of time (e.g., 60 seconds) in a case where the SCR discriminative threshold is fixed. The graph of a signal indicating the number of SCR impulse responses extracted from the wrist EDA signal (hereinafter, referred to as a wrist SCR signal) is shown as the solid line. The graph of a signal indicating the number of SCR impulse responses extracted from the finger tip EDA signal (hereinafter, referred to as a finger tip SCR signal) is shown as the dotted line. The horizontal axis indicates time (seconds), the left vertical axis indicates a wrist SCR signal (the number of signals is used as the unit), and the right vertical axis indicates a finger tip SCR signal (the number of signals is used as the unit).

C of FIG. 6 shows a graph of the SCR signal in a case where the SCR discriminative threshold is adjusted on the basis of the curvature of the SCL signal. The graph of the wrist SCR signal is shown as the solid line and the graph of the finger tip SCR signal is shown as the dotted line. It should be noted that the SCR discriminative threshold with respect to the finger tip SCR signal is a fixed value. The horizontal axis indicates time (seconds), the left vertical axis indicates a wrist SCR signal (the number of signals is used as the unit), and the right vertical axis indicates a finger tip SCR signal (the number of signals is used as the unit).

As shown in B of FIG. 6, in a case where the SCR discriminative threshold is fixed, the detection delay of the conscious reaction of the wrist SCR signal with respect to the finger tip SCR signal is 115.9 seconds.

On the other hand, as shown in C of FIG. 6, in a case where the SCR discriminative threshold is adjusted on the basis of the curvature of the SCL signal, the detection delay of the conscious reaction of the wrist SCR signal with respect to the finger tip SCR signal is 18.9 seconds.

In this manner, the SCR discriminative threshold is suitably adjusted and the detection sensitivity of the SCR is suitably adjusted. The detection delay of the conscious reaction is thus suppressed.

### 2. MODIFIED EXAMPLES

Hereinafter, modified examples of the above-mentioned embodiment of the present technology will be described.

### Modified Examples Related to Method of Setting SCR Discrimination Threshold

First of all, a modified example related to the method of setting the SCR discriminative threshold will be described.

### First Modified Example of Method of Setting SCR Discrimination Threshold

For example, the detection parameter setting unit 55 may set the SCR discriminative threshold on the basis of a distribution of impulse amplitudes of the impulse response row.

For example, in a case where the curvature of the reference SCL signal in the previous curvature determination period is negative, the detection parameter setting unit 55 sets the SCR discriminative threshold so that impulse responses of the top x % from the higher impulse amplitude in the distribution of the impulse amplitudes of the impulse response row are discriminated as the SCR impulse response.

On the other hand, for example, in a case where the curvature of the reference SCL signal in the previous curvature determination period is positive or zero, the detection parameter setting unit 55 sets the SCR discriminative threshold so that impulse responses of the top y % from the higher impulse amplitude in the distribution of the impulse amplitudes of the impulse response row are discriminated as the SCR impulse response.

It should be noted that x %>y % is set. For example, x=20% is set and y=5% is set.

Thus, in a case where the curvature of the reference SCL signal is negative, the impulse responses with a lower amplitude can be discriminated as the SCR impulse response and the detection sensitivity of the SCR increases. On the other hand, in a case where the curvature of the reference SCL signal is positive or zero, the impulse responses with a higher amplitude can be easily discriminated as the noise impulse response and the detection sensitivity of the SCR lowers.

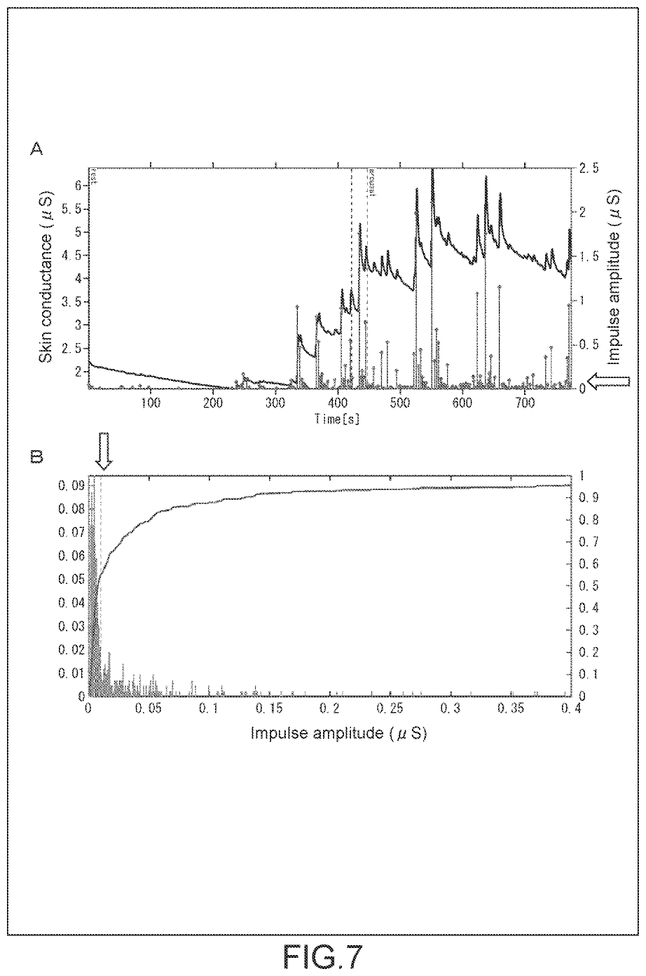

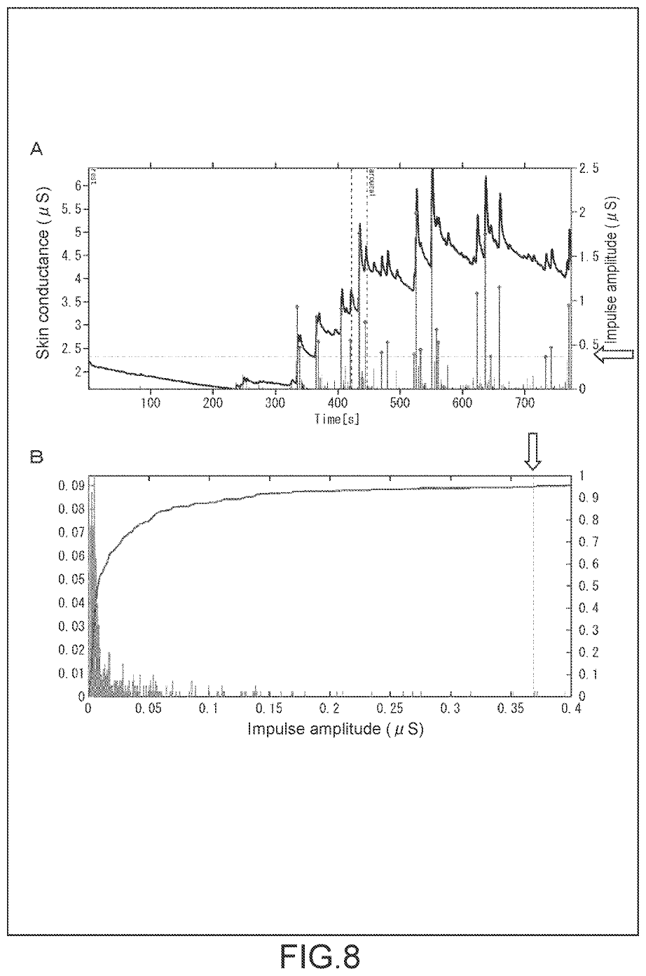

Here, a specific example of the method of setting the SCR discriminative threshold will be described with reference to FIGS. 7 and 8.

FIG. 7 shows an example in a case where the SCR discriminative threshold is fixed to be 0.01 μS. FIG. 8 shows an example in a case where the SCR discriminative threshold is set on the basis of a distribution of impulse amplitudes of impulse responses.

A of FIG. 7 and A of FIG. 8 are graphs each showing an example of the same EDA signal and an impulse response row extracted from the EDA signal. The horizontal axis indicates time (seconds), the left vertical axis indicates skin conductance (μS) of the EDA signal, and the right vertical axis indicates impulse amplitudes (μS) of the impulse response row.

B of FIG. 7 and B of FIG. 8 are graphs each showing an example of a histogram and a cumulative distribution function of the impulse amplitudes of the impulse response row in A of FIG. 7 and A of FIG. 8. The horizontal axis indicates impulse amplitudes (μS) in B of FIG. 7 and B of FIG. 8, the left vertical axis indicates a value of relative frequency probability with respect to each impulse amplitude, and the right vertical axis indicates a value of the cumulative distribution function.

The arrows in A and B of FIG. 7 each indicates the position of the SCR discriminative threshold.

FIG. 8 shows an example in a case where the curvature of the reference SCL signal in the previous curvature determination period is positive or zero and the SCR discriminative threshold is set so that impulse responses of the top 5% from the higher impulse amplitude are discriminated as the SCR impulse response. In this case, an impulse amplitude when the value of the cumulative distribution function of the impulse amplitudes is 0.95 is set as the SCR discriminative threshold. In this example, the SCR discriminative threshold is set to be 0.37. The arrows in A of FIG. 8 and B of FIG. 8 each indicate the position of the SCR discriminative threshold.

The SCR discriminative threshold is suitably set in this manner on the basis of the curvature of the reference SCL signal in the previous curvature determination period and the distribution of the impulse amplitudes of the impulse response row.

That is, in a case where the curvature of the reference SCL signal in the previous curvature determination period is negative, the SCR discriminative threshold is lowered so that much more impulse responses are discriminated as the SCR impulse response. Accordingly, the detection sensitivity of the SCR increases, and the detection delay of the conscious reaction is suppressed.

On the other hand, in a case where the curvature of the reference SCL signal in the previous curvature determination period is positive or zero, the SCR discriminative threshold is increased so that the noise impulse response is reliably cancelled. Accordingly, the detection sensitivity of the SCR lowers, noise of the SCR signal is reduced, and the detection accuracy of the conscious reaction is enhanced.

Moreover, since the SCR discriminative threshold is set on the basis of the distribution of the impulse amplitudes of the impulse response row, the noise impulse response can be more reliably cancelled, and the detection accuracy of the conscious reaction is enhanced.

### Second Modified Example of Method of Setting SCR Discrimination Threshold

Hereinabove, the example in which the SCR discriminative threshold is set on the basis of the curvature of the reference SCL signal has been described. However, the SCR discriminative threshold may be set on the basis of a descriptive variable other than the curvature of the reference SCL signal.

For example, in a case where a conscious reaction has occurred just after the period in which no SCR is generated, the impulse amplitude of the SCR impulse response decreases and the detection delay of the conscious reaction more easily occurs.

In this regard, for example, the detection parameter setting unit 55 sets the SCR discriminative threshold on the basis of an occurrence status of the SCR. For example, in a case where no SCR impulse response has been detected in the previous predetermined period of time (e.g., 60 seconds), the detection parameter setting unit 55 sets the SCR discriminative threshold to be smaller.

Accordingly, the detection sensitivity of the SCR increases in the period in which no SCR is generated, and the detection delay of the conscious reaction which occurs just after the period in which no SCR is generated is suppressed.

For example, in a case where the conscious reaction has occurred when a level of the SCL signal is low, the impulse amplitude of the SCR impulse response decreases and the detection delay of the conscious reaction more easily occurs.

In this regard, for example, the detection parameter setting unit 55 sets the SCR discriminative threshold on the basis of the level of the reference SCL signal. For example, the detection parameter setting unit 55 calculates an average value of reference SCL signals in the previous predetermined period of time (e.g., 60 seconds) as the level of the reference SCL signal. In a case where the level of the reference SCL signal is lower than a predetermined threshold, the detection parameter setting unit 55 sets the SCR discriminative threshold to be smaller than in a case where the level of the reference SCL signal is equal to or higher than the predetermined threshold. Otherwise, for example, the detection parameter setting unit 55 sets the SCR discriminative threshold to be smaller as the level of the reference SCL signal lowers and sets the SCR discriminative threshold to be larger as the level of the reference SCL signal increases.

Accordingly, the detection sensitivity of the SCR increases when the level of the reference SCL signal lowers, and the detection delay of the conscious reaction which occurs in a case where the level of the SCL signal is low is suppressed.

For example, when a rise time constant r1 of an impulse response in the impulse response row becomes larger, SCR detection failures more easily occur.

In this regard, for example, the detection parameter setting unit 55 sets the SCR discriminative threshold on the basis of the rise time constant r1 of the impulse response. For example, in a case where the rise time constant r1 is equal to or higher than a predetermined threshold, the detection parameter setting unit 55 sets the SCR discriminative threshold to be smaller than in a case where the rise time constant r1 is smaller than the predetermined threshold. For example, the detection parameter setting unit 55 sets the discriminative threshold to be smaller as the rise time constant r1 increases, and sets the SCR discriminative threshold to be larger as the rise time constant r1 decreases.

Accordingly, when the rise time constant r1 of the impulse response increases, the detection sensitivity of the SCR increases, and SCR detection failures are reduced.

For example, the possibility that the EDA signal contains noise becomes higher as the user's body movement increases.

In this regard, for example, the detection parameter setting unit 55 sets the SCR discriminative threshold on the basis of acceleration of the wearable device 1 (in vicinity of the user's wrist) acquired by the environment information detection unit 13. For example, in a case where the acceleration of the wearable device 1 is equal to or higher than a predetermined threshold, the detection parameter setting unit 55 sets the SCR discriminative threshold to be larger as compared than in the acceleration of the wearable device 1 is smaller than the predetermined threshold. Otherwise, for example, the detection parameter setting unit 55 sets the SCR discriminative threshold to be larger as the acceleration of the wearable device 1 increases, and sets the SCR discriminative threshold to be smaller as the acceleration of the wearable device 1 decreases.

Accordingly, when the acceleration of the wearable device 1 increases as the user's body movement becomes larger, the detection sensitivity of the SCR lowers and SCR impulse response detection errors are reduced. As a result, noise inclusion in the SCR signal can be suppressed, and for example, the detection accuracy of the conscious reaction is enhanced.

For example, in a case where HR or HRV absolute value of the user or a change in HR or HRV is larger, the SCR occurrence probability becomes higher.

In this regard, for example, the detection parameter setting unit 55 sets the SCR discriminative threshold on the basis of a parameter based on at least one of the HR or HRV of the user. For example, in a case where the HR or HRV absolute value of the user or the absolute value of the change rate of the HR or HRV is equal to or higher than a predetermined threshold, the detection parameter setting unit 55 sets the SCR discriminative threshold to be smaller than in a case where it is smaller than the predetermined threshold. Otherwise, for example, the detection parameter setting unit 55 sets the SCR discriminative threshold to be smaller as the HR or HRV absolute value of the user or the absolute value of the change rate of the HR or HRV increases, and sets the SCR discriminative threshold to be larger as the HR or HRV absolute value of the user or the absolute value of the change rate of the HR or HRV decreases.

Accordingly, when the change in HR or HRV of the user increases, the detection sensitivity of the SCR increases, and the detection delay of the conscious reaction is suppressed.

It should be noted that the above-mentioned methods of setting the SCR detection threshold can be combined depending on needs.

### Modified Example Related to Output Signal

Next, a modified example related to the output signal will be described.

For example, the signal processing unit 57 may estimate an internal status (e.g., conscious reaction) of the user on the basis of the SCR impulse response row and generate and output an output signal indicating an estimation result of the internal status of the user.

For example, in a case where the SCR discriminative threshold is changed on the basis of the curvature of the reference SCL signal and the like as described above, the detection sensitivity of the SCR changes before and after the SCR discriminative threshold is changed. Therefore, when the SCR discriminative threshold is changed, the discrimination result of the impulse response can suddenly change and the number of detections of the SCR impulse response can change unnaturally (e.g., discontinuously).

In this regard, for example, the signal processing unit 57 may smooth the discrimination result of the impulse response in a predetermined period of time (e.g., 30 seconds) after the SCR discriminative threshold is changed (hereinafter, referred to as a transition period).

For example, the signal processing unit 57 may smooth, during the transition period, the discrimination result of the impulse response based on the SCR discriminative threshold before change and the discrimination result of the impulse response based on the changed SCR discriminative threshold. For example, the signal processing unit 57 calculates, during the transition period, an average of the number of SCR impulse responses extracted on the basis of the SCR discriminative threshold before change and the number of SCR impulse responses extracted on the basis of the changed SCR discriminative threshold. Then, the signal processing unit 57 may generate and output an output signal indicating the calculated number of SCR impulse responses.

### Other Modified Examples

For example, the present technology can also be applied in a case of measuring an EDA at a site other than the user's wrist.

For example, the present technology can also be applied in a case of detecting physiological indicators other than the SCR and the SCL. For example, the present technology can also be applied in a case of detecting skin potential reflex (SPR) or a skin potential level (SPL) on the basis of the EDA signal based on a skin potential of the user.

For example, the present technology can also be applied in a case of setting a detection parameter other than the SCR discriminative threshold.

For example, a coefficient of second-degree terms when fitted to a quadratic function of the SCL signal can be used as the curvature of the SCL signal.

Detection processing of physiological indicators such as the SCR and the SCL does not necessarily need to be executed in the wearable device 1. For example, an information processing terminal such as a smartphone, a tablet terminal, a mobile phone, and a personal computer, or an external apparatus such as a server may execute detection processing of the physiological indicators. In this case, for example, all or some of the functions of the control unit 15 in FIG. 2 are provided in the external apparatus, and the external apparatus executes the detection processing of the physiological indicators while communicating with the wearable device 1 on line.

### 3. SPECIFIC EXAMPLES OF EFFECTS OF PRESENT TECHNOLOGY

Next, specific examples of effects of the present technology will be described.

As described above, in accordance with the present technology, the wearable device 1 is capable of accurately detecting physiological indicators of the user in a state in which it is attached to a site to which the wearable device 1 can be easily attached, such as the wrist, without interfering with the user's movement. Moreover, the wearable device 1 or an apparatus at the subsequent stage of the wearable device 1 is capable of accurately estimating the internal status of the user on the basis of the physiological indicators of the user.

Therefore, the present technology can be effectively applied to various applications using physiological indicators and internal statuses of the user, such as health care, game, and sports.

For example, in accordance with the present technology, times of sweating actions for predicting attacks to an epileptic patient can be accurately detected in the field of health care.

For example, in accordance with the present technology, internal statuses of the user (e.g., impatience, astonishment, conscious reaction, etc.) can be estimated and fed back in real time in the field of games, and an experience according to the internal statuses of the user can be provided in real time.

For example, in accordance with the present technology, a time at which sweating is started can be correctly detected in a case of measuring the amount of sweating during a training in the field of sports, and effects and the like of the training can be correctly analyzed, and a plan and the like of the training can be fed back.

For example, in accordance with the present technology, a time at which stress is generated can be correctly detected in a case of daily estimating a stress status in the field of mental care.

For example, in accordance with the present technology, a cognitive load status of a student can be correctly detected in the field of education.

For example, in accordance with the present technology, an excitement status when the user experiences an entertainment can be correctly assessed without interfering with an environment where the user enjoys the entertainment in the field of entertainment.

### 4. OTHERS

The above-mentioned series of processing may be executed by hardware or may be executed by software. If the series of processing is executed by software, programs that configure the software are installed in a computer. Here, the computer includes a computer incorporated in dedicated hardware or a general-purpose personal computer for example, which is capable of executing various functions by installing various programs.

Programs executed by the computer can be, for example, provided recorded on the removable medium that is a package medium or the like. Moreover, the programs can be provided via a wired or wireless transmission medium such as a local area network, the Internet, and digital satellite broadcasting.

It should be noted that the programs executed by the computer may be programs processed chronologically in the order described in the present specification or may be programs processed concurrently or at a required time, e.g., upon calling.

Moreover, in the present specification, the system means a set of a plurality of components (apparatuses, modules (parts), etc.) and it does not matter whether or not all the components are contained in the same casing. Therefore, a plurality of apparatuses housed in separate casings and connected via a network and a single apparatus including a plurality of modules housed in the same casing are both considered as the system.

In addition, embodiments of the present technology are not limited to the above-mentioned embodiments and various modifications can be made without departing from the gist of the present technology.

For example, the present technology can take a cloud computing configuration in which a plurality of apparatuses shares and cooperatively processes a single function via a network.

Moreover, a plurality of apparatuses can share and execute the respective steps described above with reference to the above-mentioned flowcharts rather than executing them by a single apparatus.

In addition, if a single step is constituted by a plurality of processes, a plurality of apparatuses can share and execute the plurality of processes of the single step rather than executing them by a single apparatus.

### Combination Examples of Configurations

The present technology can also take the following configuration.

(1) An information processing apparatus, including:
a detection parameter setting unit that sets a detection parameter on the basis of at least one of a feature amount of an electrodermal activity signal indicating an electrodermal activity of a predetermined site of a user, biological information of the user, or environment information of the user, the detection parameter being used in a case of detecting a physiological indicator on the basis of the electrodermal activity signal; and
a physiological indicator detection unit that detects the physiological indicator on the basis of the electrodermal activity signal and the detection parameter.

(2) The information processing apparatus according to (1), in which
the electrodermal activity signal indicates skin conductance of the site,
the physiological indicators includes a skin conductance response (skin conductance activity), and
the detection parameter includes a discriminative threshold used for discriminating the skin conductance response.

(3) The information processing apparatus according to (2), further including
an impulse response extraction unit that extracts an impulse response of the electrodermal activity signal, in which
the discriminative threshold is a threshold for discriminating whether or not the impulse response is a skin conductance response impulse response due to the skin conductance response on the basis of an amplitude of the impulse response.

(4) The information processing apparatus according to (3), in which
a feature amount of the electrodermal activity signal includes at least one of a curvature of a skin conductance level signal indicating a skin conductance level included in the electrodermal activity signal, a level of the skin conductance level signal, an occurrence status of the skin conductance response in the electrodermal activity signal, or a rise time constant of the impulse response,
the biological information includes at least one of a heart rate or heart rate variability of the user, and
the environment information includes at least one of acceleration in vicinity of the site, angular velocity in vicinity of the site, audio data, image data, or information regarding an action of the user.

(5) The information processing apparatus according to (4), in which
the detection parameter setting unit sets the discriminative threshold so as to increase detection sensitivity of the skin conductance response in a case where the curvature of the skin conductance level signal is negative.

(6) The information processing apparatus according to (4) or (5), in which
the detection parameter setting unit sets the discriminative threshold so as to increase detection sensitivity of the skin conductance response in a case where the level of the skin conductance level signal is smaller than a predetermined threshold.

(7) The information processing apparatus according to any of (4) to (6), in which
the detection parameter setting unit sets the discriminative threshold so as to increase detection sensitivity of the skin conductance response in a case where the skin conductance response is not generated in the electrodermal activity signal.

(8) The information processing apparatus according to any of (4) to (7), in which
the detection parameter setting unit sets the discriminative threshold so as to increase detection sensitivity of the skin conductance response in a case where the rise time constant of the impulse response is equal to or higher than a predetermined threshold.

(9) The information processing apparatus according to any of (4) to (8), in which
the detection parameter setting unit sets the discriminative threshold so as to lower detection sensitivity of the skin conductance response in a case where the acceleration in vicinity of the site is equal to or higher than a predetermined threshold.

(10) The information processing apparatus according to any of (4) to (9), in which
the detection parameter setting unit sets the discriminative threshold on the basis of a parameter based on at least one of the heart rate or the heart rate variability of the user.

(11) The information processing apparatus according to any of (3) to (10), in which
the detection parameter setting unit sets the discriminative threshold on the basis of a distribution of amplitudes of the impulse response.

(12) The information processing apparatus according to any of (3) to (11), further including
a signal processing unit that performs smoothing of the number of skin conductance response impulse responses extracted on the basis of the discriminative threshold before change and the number of skin conductance response impulse responses extracted on the basis of the changed discriminative threshold, the smoothing being performed in a predetermined period of time after the discriminative threshold is changed.

(13) The information processing apparatus according to any of (3) to (12), further including
a signal processing unit that generates, on the basis of the extracted skin conductance response impulse response, a skin conductance response signal indicating the skin conductance response included in the electrodermal activity signal.

(14) The information processing apparatus according to any of (1) to (13), further including
the site is a wrist of the user.

(15) An information processing method, including:
setting a detection parameter on the basis of at least one of a feature amount of an electrodermal activity signal indicating an electrodermal activity of a predetermined site of a user, biological information of the user, or environment information of the user, the detection parameter being used in a case of detecting a physiological indicator on the basis of the electrodermal activity signal; and
detecting the physiological indicator on the basis of the electrodermal activity signal and the detection parameter.

(16) A program that causes a computer to execute processing of:
setting a detection parameter on the basis of at least one of a feature amount of an electrodermal activity signal indicating an electrodermal activity of a predetermined site of a user, biological information of the user, or environment information of the user, the detection parameter being used in a case of detecting a physiological indicator on the basis of the electrodermal activity signal; and
detecting the physiological indicator on the basis of the electrodermal activity signal and the detection parameter.

It should be noted that the effects set forth herein are merely exemplary and not limitative, and other effects may be provided.

### REFERENCE SIGNS LIST

1 wearable device
11 sweat sensor
12 biological information detection unit
13 environment information detection unit
15 control unit
52 impulse response extraction unit
53 reference signal generation unit
54 feature amount extraction unit
55 detection parameter setting unit
56 physiological indicator detection unit
57 signal processing unit

What is claimed is:

1. An information processing apparatus, comprising:
a detection parameter setting unit that sets a detection parameter on a basis of at least one of a feature amount of an electrodermal activity signal indicating an electrodermal activity of a predetermined site of a user, biological information of the user, or environment information of the user, the detection parameter being used in a case of detecting a physiological indicator on a basis of the electrodermal activity signal; and
a physiological indicator detection unit that detects the physiological indicator on a basis of the electrodermal activity signal and the detection parameter.

2. The information processing apparatus according to claim 1, wherein
the electrodermal activity signal indicates skin conductance of the site,
the physiological indicators includes a skin conductance response (skin conductance activity), and
the detection parameter includes a discriminative threshold used for discriminating the skin conductance response.

3. The information processing apparatus according to claim 2, further comprising
an impulse response extraction unit that extracts an impulse response of the electrodermal activity signal, wherein
the discriminative threshold is a threshold for discriminating whether or not the impulse response is a skin conductance response impulse response due to the skin conductance response on a basis of an amplitude of the impulse response.

4. The information processing apparatus according to claim 3, wherein
a feature amount of the electrodermal activity signal includes at least one of a curvature of a skin conductance level signal indicating a skin conductance level included in the electrodermal activity signal, a level of the skin conductance level signal, an occurrence status of the skin conductance response in the electrodermal activity signal, or a rise time constant of the impulse response,
the biological information includes at least one of a heart rate or heart rate variability of the user, and
the environment information includes at least one of acceleration in vicinity of the site, angular velocity in vicinity of the site, audio data, image data, or information regarding an action of the user.

5. The information processing apparatus according to claim 4, wherein
the detection parameter setting unit sets the discriminative threshold so as to increase detection sensitivity of the skin conductance response in a case where the curvature of the skin conductance level signal is negative.

6. The information processing apparatus according to claim 4, wherein
the detection parameter setting unit sets the discriminative threshold so as to increase detection sensitivity of the skin conductance response in a case where the level of the skin conductance level signal is smaller than a predetermined threshold.

7. The information processing apparatus according to claim 4, wherein
the detection parameter setting unit sets the discriminative threshold so as to increase detection sensitivity of the skin conductance response in a case where the skin conductance response is not generated in the electrodermal activity signal.

8. The information processing apparatus according to claim 4, wherein
the detection parameter setting unit sets the discriminative threshold so as to increase detection sensitivity of the skin conductance response in a case where the rise time constant of the impulse response is equal to or higher than a predetermined threshold.

9. The information processing apparatus according to claim 4, wherein
the detection parameter setting unit sets the discriminative threshold so as to lower detection sensitivity of the skin conductance response in a case where the acceleration in vicinity of the site is equal to or higher than a predetermined threshold.

10. The information processing apparatus according to claim 4, wherein
the detection parameter setting unit sets the discriminative threshold on a basis of a parameter based on at least one of the heart rate or the heart rate variability of the user.

11. The information processing apparatus according to claim 3, wherein
the detection parameter setting unit sets the discriminative threshold on a basis of a distribution of amplitudes of the impulse response.

12. The information processing apparatus according to claim 3, further comprising
a signal processing unit that performs smoothing of the number of skin conductance response impulse responses extracted on a basis of the discriminative threshold before change and the number of skin conductance response impulse responses extracted on a basis of the changed discriminative threshold, the smoothing being performed in a predetermined period of time after the discriminative threshold is changed.

13. The information processing apparatus according to claim 3, further comprising
a signal processing unit that generates, on a basis of the extracted skin conductance response impulse response, a skin conductance response signal indicating the skin conductance response included in the electrodermal activity signal.

14. The information processing apparatus according to claim 1, further comprising
the site is a wrist of the user.

15. An information processing method, comprising:
setting a detection parameter on a basis of at least one of a feature amount of an electrodermal activity signal indicating an electrodermal activity of a predetermined site of a user, biological information of the user, or environment information of the user, the detection parameter being used in a case of detecting a physiological indicator on a basis of the electrodermal activity signal; and
detecting the physiological indicator on a basis of the electrodermal activity signal and the detection parameter.

16. A program that causes a computer to execute processing of:
setting a detection parameter on a basis of at least one of a feature amount of an electrodermal activity signal indicating an electrodermal activity of a predetermined site of a user, biological information of the user, or environment information of the user, the detection parameter being used in a case of detecting a physiological indicator on a basis of the electrodermal activity signal; and
detecting the physiological indicator on a basis of the electrodermal activity signal and the detection parameter.
